(19) United States
(12) Patent Application Publication
(10) Pub. No.: US 2004/0115208 A1
(43) Pub. Date: Jun. 17, 2004

(54) Method of using colloidal metal-protein composition for treatment of cancer

(76) Inventors: Frank Q. Li, Montgomery Village, MD (US); Wan-Ching Lai, Rockville, MD (US); Yong Liang Chu, Rockville, MD (US)

Correspondence Address: Supervisor, Patent Prosecution Services, PIPER RUDNICK LLP, 1200 Nineteenth Street, N.W., Washington, DC, 20036-2412, US

(21) Appl. No.: 10/319,556

(22) Filed: Dec. 16, 2002

(51) Int. Cl. A61K 39/395; A61K 33/24; A61K 38/19

(52) U.S. Cl. 424/178.1; 424/617; 514/6; 424/85.1

(57) ABSTRACT

The present invention relates to methods of using compositions for stimulate an immunological response against cancer. The method comprising contacting colloidal metal with a tumor cell in an aqueous solution to form a colloidal metal-protein complex, separating the colloidal metal-protein complex from other cellular components, and administering a therapeutically effective amount of the colloidal metal-protein complexes to a tumor-bearing subject. The present invention further relates to a composition which is made by contacting colloidal metal with a cell in an aqueous solution to form a colloidal metal-protein complex, and separating the colloidal metal-protein complex from other cellular components.

### FIELD OF THE INVENTION

The present invention is directed to methods and compositions for the enhancement of immune responses in a mammal. These methods and compositions are especially useful in the development of vaccines thereof and further useful in stimulating immunological responses against cancers.

### BACKGROUND OF THE INVENTION

A number of approaches have been utilized in treating malignant growth of tissue cells such as tumor cells. One of the approaches is to induce or enhance host immune responses against the tumor cells. A major problem in developing an anti-tumor immune response is the immune recognition of tumor cells. Tumor cells are genetically altered normal cells. The immune system must be able to recognize tumor cells as non-self in order to mount an effective immune response against these cells. There is a widespread agreement that tumor cells express tumor-specific antigens (TSAs) due to genetic changes during malignant transformation and tumor progression. The TSAs, when properly presented by antigen presenting cells (APCs), may be recognized by the immune system and lead to a tumor-specific immune response. However, malignant tumor cells often develop mechanisms, such as the down regulation of certain co-stimulation molecules on tumor cell surface, that either prevent the development of tumor-specific immune responses or induce immune tolerance to the tumor cells.

It has been found that various cytokines, such as TNFα and IL-2, are capable of stimulating an efficacious immune response against tumors. The problem with the cytokine treatment, however, is that cytokines often cause severe side effects and exhibit unwanted toxicity when injected into a human or animal. In U.S. Pat. Nos. 6,274,552 and 6,407,218, Tamarkin et al. generally describe the use of colloidal gold to reduce or eliminate the in vivo toxicity of certain biologically-active factors, such as TNFα and IL-2, and thereby allowing the factors to exert their therapeutic effects. The methods, however, require conjugation of colloidal gold to proteins isolated and purified from natural sources or genetically engineered materials.

Another approach to eliciting an anti-tumor immune response is to immunize a patient using the patient's own tumor cells (“self-vaccination”). This approach is based on the hypothesis that immune tolerance to tumor-specific antigens could be broken or superceded more easily than tolerance to self-antigens. Hence, it is possible to use self vaccination to induce immune response to tumor-specific antigens that are not expressed by normal cells.

A series of recent experimental findings have led to a shift from unique tumor-specific antigens to tissue-specific antigens as promising target for immune therapy [for a general review on this subject, see Pardoll D M Proc. Natl. Acad. Sci. USA 96:5340-5342, (1999)]. It was found that melanoma-specific CD8⁺ T cells recognized melanocyte-specific antigens rather than melanoma-specific antigens. Most of these melanocyte-specific antigens are involved in normal melanin biosynthesis. Further studies revealed that these melanocyte-specific antigens represented the dominant target for melanoma-specific CD8⁺ T cells, while recognition of melanoma-specific peptides represented only infrequent reactivities among melanoma-reactive CD8⁺ T cells. These findings created an important linkage between anti-tumor immune response and autoimmune response and raised the possibility of treating tumor by breaking self-tolerance to tissue-specific antigens, i.e., the induction of an autoimmune response.

### SUMMARY OF THE INVENTION

The present invention relates to a method for prevention and treatment of tumor in a human, said method comprising the steps of contacting colloidal metal with tumor cells in an aqueous solution, wherein said tumor cells are isolated from said human, and wherein said colloidal metal bind to cells membrane protein to form colloidal metal-protein complexes, separating said colloidal metal-protein complexes from other cellular components, and administering a therapeutically effective amount of said colloidal metal-protein complexes to said human. The present invention further relates to a composition for stimulating an immune response to tumors in a mammal, said composition is made by contacting colloidal metal with a cell in an aqueous solution wherein said colloidal metal binds to cell membrane proteisn to form a colloidal metal-protein complex, and separating said colloidal metal-protein complex from other cellular components.

### BRIEF DESCRIPTION OF THE FIGURES

FIG. 1 illustrates the interaction between colloidal gold and a membrane protein on a cell in suspension.

FIG. 2 is a general flowchart illustrating a method by which a colloidal gold-membrane protein complex is formed.

FIG. 3 shows the immunoreactivity of gold-rat IL-6 mAb complex.

FIGS. 4A and 4B illustrate the gold-MSA-induced mouse immune response to MSA.

FIG. 4A shows the result of an ELISA using sera from MSA and gold-MSA immunized mice and MSA coated assay plate.

FIG. 4B shows the result of an ELISA using sera from MSA and gold-MSA immunized mice and gold-MSA coated assay plate.

FIGS. 5A, 5B and 5C illustrate the effect of gold-P815 immunization on IFNγ secretion from mouse spleen cells.

FIG. 5A shows IFNγ secretion of spleen cells from mice immunized with KLH+P815 cells after coincubation with P815 cells.

FIG. 5B shows IFNγ secretion of spleen cells from mice immunized with KLH+gold-P815 complex after coincubation with P815 cells.

FIG. 5C shows IFNγ secretion of spleen cells from mice immunized with HA+gold-P815 complex after coincubation with P815 cells.

### DETAILED DESCRIPTION OF THE INVENTION

The majority of tumor antigens are self-antigens that are derived from and expressed by the normal cells. Frequently, the tumor antigen is identical to the normal antigen though it is expressed at higher levels or endowed with a negligible mutation insufficient for its distinction from the self-antigen. One of the escape mechanisms of malignant cells from the immune system is their similarity to their normal counterpart resulting in their low visibility by the immune system.

One aspect of the present invention relates to a method for treating tumor by immunizing a tumor bearing mammal with a colloidal metal-protein complex prepared by incubating colloidal metal with the tumor cells derived from the mammal.

Another aspect of the present invention relates to a colloidal metal-protein composition which is made by (a) contacting colloidal metal to a cell, therefore allowing the colloidal metal binds to a cell membrane protein to form a colloidal metal-protein complex, and (b) separating the colloid metal-protein complex from the other cellular components. Such a composition can be used to enhance an immune response in a mammal and, in particular, to enhance an anti-tumor immune response in a cancer patient.

Another aspect of the present invention relates to methods for prevention and treatment of cancer by administering an effective amount of the colloidal metal-protein composition to a patient.

In order to provide a clear and consistent understanding of the specification and claims, including the scope given to such claims, the following definitions are provided:

The term “cell membrane protein,” as used herein, is any protein associated with a cellular membrane, including proteins having an extracellular domain and proteins situated on the surface, or in the lipid bi-layer, of the cell membrane. The proteins may be glycoproteins. Preferably, the proteins are surface antigens of a tumor cell. The cellular membrane may be that of a single cell, such as from a multicellular organism, more preferably a mammalian cell, and most preferably a tumor cell.

The term “a therapeutic effective amount,” as used herein, is that amount capable of achieving the desired effect. The effective amount is an effective amount within each encapsulated structure that is sufficient to produce the desired response. As described above, an effective amount can be an amount sufficient to prime, induce or potentiate an immune, for example, bound protein concentrations range from 1 ng to 50 mg per immunization. Immunization can be given, but not limited to, one to six times.

The term “non-self” antigens are those antigens on substances entering a subject, or exist in a subject but are detectably different or foreign from the subject's own constituents, whereas “self” antigens are those which, in the healthy subject, are not detectably different or foreign from its own constituents. However, under certain conditions, including in certain disease states, an individual's immune system will identify “non-self” antigens as its own constituents as “self,” and will not initiate an immune response against “non-self”. Conversely, an individual's immune system may also identify “self” antigens as “non-self,” and mount an immune response against the “self” antigens, leading to auto-immune diseases.

“Tumor-specific antigen(s)” refers to antigens that are present only in a tumor cell at the time of tumor development in a subject. For example, a melanoma-specific antigen is an antigen that is expressed only in melanoma cells but not in normal melanocytes.

“Tissue-specific antigen(s)” refers to antigens that are present only in certain kind of tissues at a certain time in a subject. For example, a melanocyte-specific antigen is an antigen that is expressed in all melanocytes, including normal melanocytes and abnormal melanocytes (melanoma cells).

The term “colloidal metal,” as used herein, includes any water-insoluble metal particle or metallic compound dispersed in liquid water. Colloidal metal may include the following metals and in all of their various oxidation states: gold, silver, platinum, titanium, vanadium, chromium, manganese, cobalt, gallium, strontium, tungsten, rhenium, gadolinium, aluminum, ruthenium, zinc, iron, nickel and calcium. Other suitable metals may also include the following in all of their various oxidation states: lithium, sodium, magnesium, potassium and scandium. The metals are preferably provided in ionic form.

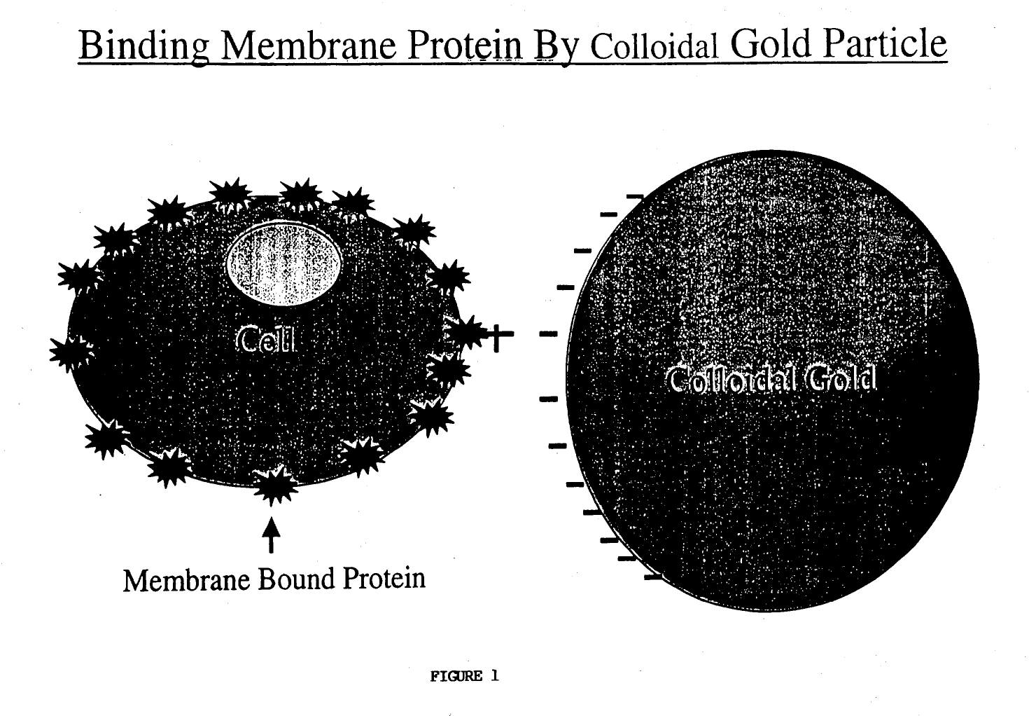

As shown in FIG. 1, negatively charged colloidal gold can form complexes with positively charged membrane proteins through electrostatic interactions. It has been hypothesized that the protein structure is slightly changed when bound to a colloidal gold. This structure change may alter the immunogenicity of the membrane protein, leading to enhanced immune responses to the protein or abrogation of an exiting tolerance to the protein, when the colloidal metal-protein complex is introduced into a mammalian subject. The colloidal metal-protein complex is formed by incubating the colloidal gold with a cell suspension.

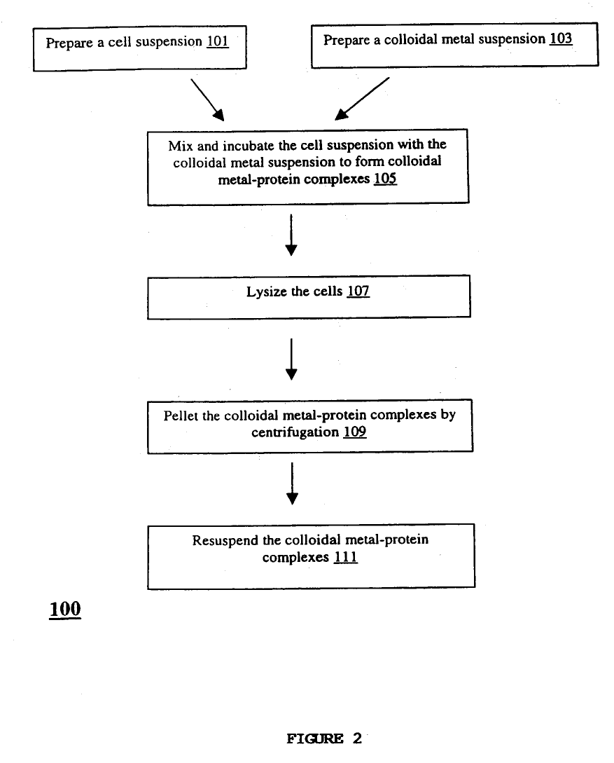

FIG. 2 provides a flowchart illustrating an embodiment (100) of a method by which the colloid metal-protein complex is formed.

Briefly, a cell suspension is prepared (101) in a suitable cell suspension buffer. A colloidal gold suspension is also prepared (103) in a desired buffer. The two suspensions are mixed and incubated (105) for a period of time to allow the interaction of colloidal gold with proteins on the cell surface to form colloidal metal-protein complexes. The cells are then lysized (107) to release the colloidal metal-protein complexes, which are then precipitated by centrifugation (109) and resuspended (111) for future administration to a subject.

The colloidal golds are preferably provided in ionic form, particularly in the form of Au³⁺. An especially preferred form of colloidal gold is HAuCl₄, having a particle size of about 1-80 nm, preferably of about 30-35 nm, and most preferably of about 32 nm. A preferred suspension buffer for colloidal gold is sodium borate buffer with a pH value from 6 to 10, and most preferably from 8-9.

The amount of colloidal gold that is used in the present invention is between approximately 0.001% and 0.1%, preferably between approximately 0.01% and 0.1%.

The cells may be isolated mammalian cells such as peripheral blood cells, or cultured mammalian cells such as tumor cell lines, and primary culture of tumor or normal tissue cells. The cells may be suspended in any buffer that maintains the viability of the cells, while facilitates the formation of the colloidal metal-protein complex. Examples of the cell suspension buffer include: (phosphate buffered saline (PBS) and RPMI 1640 and DMEM. A preferred buffer is PBS.

In one embodiment, colloidal metal is incubated with tumor cells to form colloidal metal-tumor antigen complexes. The colloidal metal-tumor antigen complexes is isolated and used to immunize a tumor-bearing subject to induce anti-tumor immune response. Preferably, the tumor cells are isolated from the tumor-bearing subject. The immunization may be performed repetitively to further augment immune response.

In another embodiment, colloidal metal is incubated with tumor cells to form colloidal metal-tumor antigen complexes. The colloidal metal-tumor antigen complexes is used to immunizing a non-tumor bearing subject as a prophylactic treatment.

Another aspect of the present invention relates to a pharmaceutical composition comprising an effective amount of the colloidal metal-protein complex of the present invention and a pharmaceutically acceptable carrier. Such compositions are liquids or lyophilized or otherwise dried formulations and may further include diluents of various buffer content, (e.g., Tris-HCl, acetate, phosphate) pH and ionic strength, additives such as albumin and gelatin to prevent absorption to surfaces, detergents (e.g., Tween 20, Tween 80, Pluronic F68, bile acid salts), solubilizing agents (e.g., glycerol, polyethylene glycerol); anti-oxidants (e.g., ascorbic acid, sodium metabisulfite), preservatives (e.g. Thimerosal, benzyl alcohol, parabens), bulking substances or tonicity modifiers (e.g. lactose, mannitol).

Within certain aspects, the pharmaceutical composition of the present invention may be used as a vaccine for cancer. As used herein, vaccine means an agent used to stimulate the immune system of an animal so that protection is provided against an antigen not recognized as a self-antigen by the immune system.

Vaccines may comprise one or more such compositions and an immune enhancer. An immune enhancer may be any substance that enhances or potentiates an immune response (antibody and/or cell-mediated) to an exogenous antigen. Examples of immune enhancer include adjuvants, biodegradable microspheres (e.g., polylactic galactide) and liposomes (into which the compound is incorporated).

A vaccine may contain DNA encoding one or more immune stimulating polypeptide, such that the polypeptide is generated in situ. The DNA may be present within any of a variety of delivery systems known to those of ordinary skill in the art, including nucleic acid expression vectors, gene delivery vectors, and bacteria expression systems and viral expression systems. Numerous gene delivery techniques are well known in the art. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be “naked,” as described, for example, in Ulmer et al., Science 259:1745-1749, (1993) and reviewed by Cohen, Science 259:1691-1692, (1993). The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

Any of a variety of immune enhancers may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadellci pertussis or Mycobacterium tuberculosis derived proteins. Suitable adjuvants are commercially available as, for example, complete Freund's adjuvant (CFA) and incomplete Freund's adjuvant (IFA); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable micro spheres; monophosphoryl lipid A and quil A; keyhole limpet hemocyanin (KLH); and hyaluronic acid (HA). Cytokines, such as granulocyte macrophagecolony stimulating factor (GMCSF), codon modified GMCSF (cGMCSF) (details of the codon modification is described in the co-pending U.S. patent application Ser. No. 10/188,056, which is filed Jul. 3, 2002, and which is herein incorporated by reference), or interleukin-2, -7, or -12, may also be used as adjuvants.

Any vaccine provided herein may be prepared using well known methods that result in a combination of antigen, immune response enhancer and a suitable carrier or excipient. The compositions described herein may be administered as part of a sustained release formulation [i.e., a formulation such as a capsule, sponge or gel (composed of polysaccharides, for example) that effects a slow release of compound following administration]. Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Vaccines may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine may be stored in a freeze-dried condition requiring only the addition of a sterile liquid carrier immediately prior to use.

The present invention is also directed to a method of preventing or treating a disease in a subject using by administering a pharmaceutical composition comprising a colloidal metal-protein complex containing membrane proteins. The subject may be a human or a non-human animal such as a mouse or rat. Such non-human animals may be useful model systems for human diseases.

One embodiment of the present invention is a method for treating tumor by immunizing a tumor patient with a colloidal metal-protein complex prepared by incubating a colloidal metal with the tumor cells derived from the patient.

Immunization refers to the process of inducing a continuing high level of antibody and/or cellular immune response in which T-lymphocytes can either kill the pathogen and/or activate other cells (e.g., phagocytes) to do so in the immunized animal, which is directed against a pathogen or antigen to which the animal has been previously exposed.

In another embodiment, the colloidal metal-protein complex is administered to the patient with immune enhancers, such as KLH, GMCSF, cGMCSF or HA.

The present invention is further illustrated by the following examples, which are not to be construed in any way as imposing limitations upon the scope thereof.

### EXAMPLE 1

### Preparation of Colloidal Gold

All glassware was scrupulously clean. Glass and plastic containers and stirrers were cleaned in aqua regia and thoroughly washed in deionized water. All reagents including deionized water were filtered (0.22 μm) immediately before use.

Colloidal gold (32 nm) was prepared by rediction of chloroauric acid with sodium citrate. 2.5 ml of a 4% HAuCl₄ solution was added to 1,000 ml of deionized water. The mixture was heated under reflex, 15 ml of 1% sodium citrate was added to the mixture when the mixture start to boil. The reduction with boiling was continued for 10-15 min until the color of the solution became red. After cooling, colloidal gold solution was filtered through a 0.22 μm filter, dialyzed against 8 liters of 2 mM sodium borate (Borax buffer, pH 8.0) in a dialysis tubing with molecular cutoff of 10,000-12,000 at 4° C. overnight sterilized through 0.22 μm filter and stored at 4° C.

### EXAMPLE 2

### Preparation of Protein-Gold Complexes from Rat IL-6 Monoclonal Hybridoma Cells.

Colloidal gold (32 nm) was dialysized against borax buffer (pH 9.0) in a dialysis tubing with molecular cutoff of 10,000-12,000 at 4° C. overnight. Hybridoma cells were cultured in Debuco's modified Eagle's medium (DMEM) supplemented with 10% fetal bovine serum (FBS) and antibiotics (penicillin and streptomycin) in T-150 tissue culture flask at 37° C. and 5% CO₂ to 80% confluence. The cells were harvested and counted (the cell viability should be greater than 90%). After washing with PBS three times, the cells were mixed with dialyzed colloidal gold solution at a ratio of 10 million cells per 20 ml gold solution in a 50 ml centrifuge tube. The mixture was rocked over night at 4° C., then centrifuged at 4° C. for 10 min at 4,000 rpm. After removing the supernatant, the colloidal gold-bound cells were lysized with 10 ml of deionized water containing a cocktail of protease inhibitors (Roche, catalog No. 1836153) at 4° C. for 2 hrs. The lysate was centrifuged again at 4000 rpm for ten minutes. The supernatant was removed and the pellet (colloidal gold bound IgGs) was suspended in PBS containing 0.1% bovine serum albumin (BSA) and stored at −80° C. for ELISA.

### EXAMPLE 3

### ELISA Procedurals for Detecting Colloidal Gold Bound Mouse Antibody to Rat IL-6.

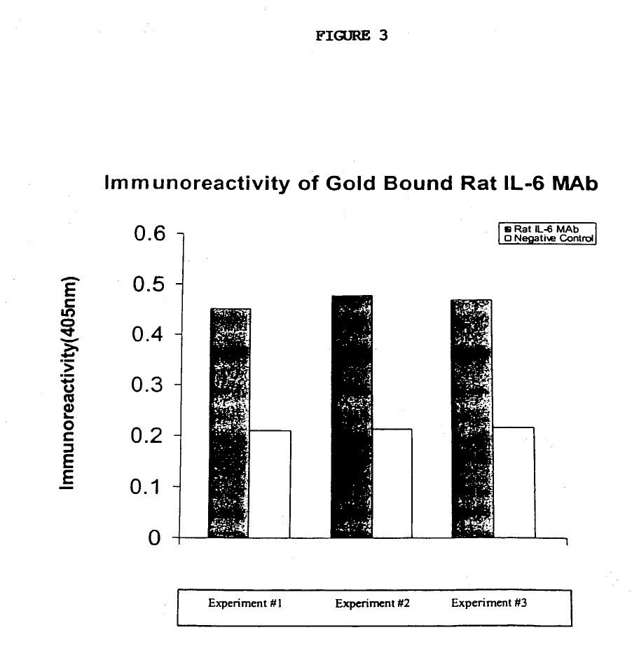

Polystyrene 96-well plate (Nunc) was coated with protein A (Sigma) at 5 μg/ml in coating buffer (Na₂CO₃ 15.9 g/l and NaHCO₃ 29.3 g/l in deionized water, pH 9.6) at 4° C. over night. The plate was washed three times with washing buffer, 0.2% Tween 20 in PBS. 200 μl of blocking buffer (2% BSA, 10% FBS and 0.1% sodium azide in tris buffered saline (TBS) was added to each well and incubated for 30 min at room temperature (RT). After washing the plate five times, 100 μl of diluted colloidal gold bound IgG (1:100 dilution in an assay diluent containing 0.1% BSA and 0.1% sodium azide in TBS) was added to each well in duplicate and incubated for 2 hrs at RT. The plate was then washed five times with washing buffer. Goat anti-mouse IgG conjugated to alkaline phosphatase (KPL) (1:1000 dilution in TBS with 1% BSA) was added to each well and incubated for 30 min at RT. After six washes with washing solution, a substrate solution (2 mg/ml p-nitrophenyl phosphate in substrate buffer containing 10% diethanolamine, 100 μg/ml MgCL₂ 6H₂O, pH 9.8) was added to each well. The result was read with a microplate reader at 405 nm and summarized in FIG. 3.

### EXAMPLE 4

### Preparation of Colloidal Gold-conjugated Mouse Serum Albumin (MSA).

MSA (Sigma) was reconstituted in deionized water to a concentration of 10 mg/ml. Colloidal gold (32 nm) suspension was concentrated by centrifugating 9 ml of original suspension (12,000 rpm in a Marathon 21000R centrifuge, Fisher Scientific) and resuspending the pellet in 0.5 ml of Borax buffer (pH 6.26). The concentrated colloidal gold (32 nm) suspension was mixed with 150 μl (1.5 mg) of MSA. The mixture was incubated at RT for 6 hrs on a rocking plateform, and then centrifuged at 12,000 rpm to separate colloidal gold-bound MSA from unbound MSA. The pellet (colloidal gold-bound MSA) was reconstituted in 1.5 ml of deionized water containing 10% polyethylene glycol (PEG, molecular weight 1450). The suspension was stored at −80° C. until immunization.

### EXAMPLE 5

### Immunization with Colloidal Gold-MSA conjugates and detection of Immune Reactivity to MSA.

Before immunization, experimental mice were bled from the tail vein with a capillary tube. Sera were isolated and stored at −20° C. as negative controls for ELISA. Each of the 10 female Balb/c mice at 6 weeks of age was immunized with either 150 μg of colloidal gold-MSA or free MSA. The MSA antigen (bound or free) was given intraperitoneally along with 100 μl of complete Freund's adjuvant (CFA) for the first challenge and with 100 μl of incomplete Freund's adjuvant (IFA) for the following boosts. After three consecutive injections (within one month), the mice were bled and the sera were tested by direct ELISA for anti-MSA antibodies.

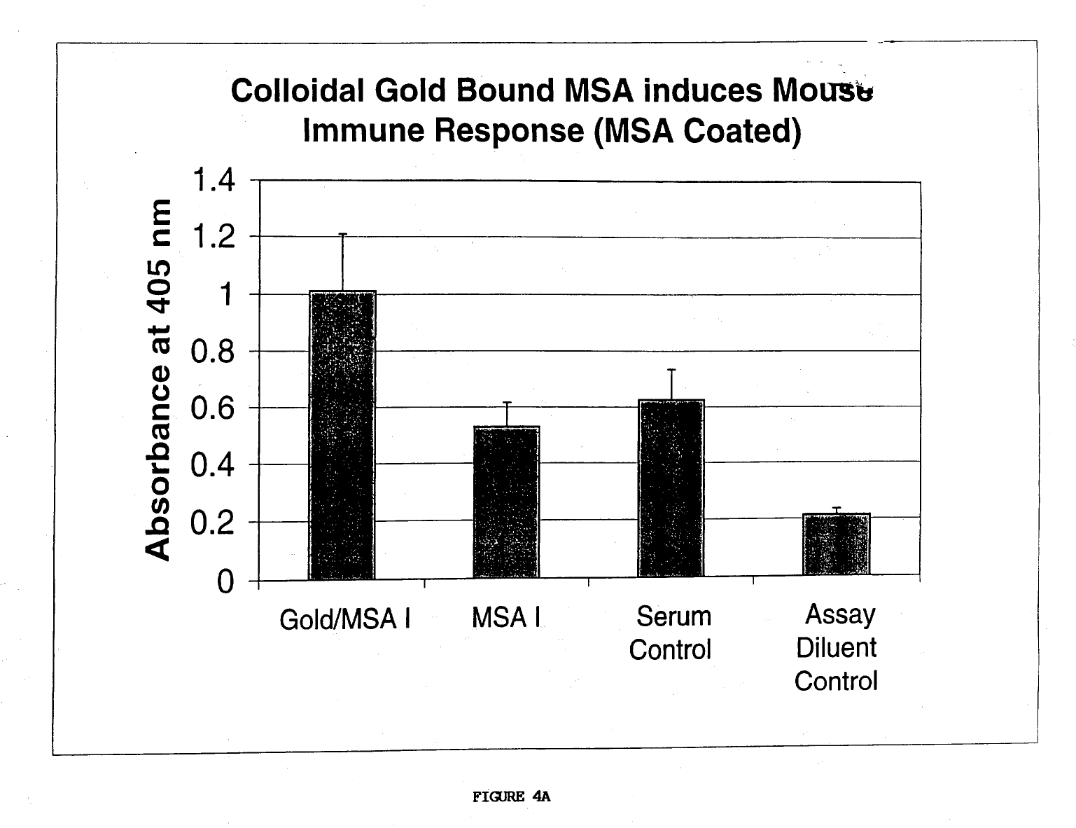

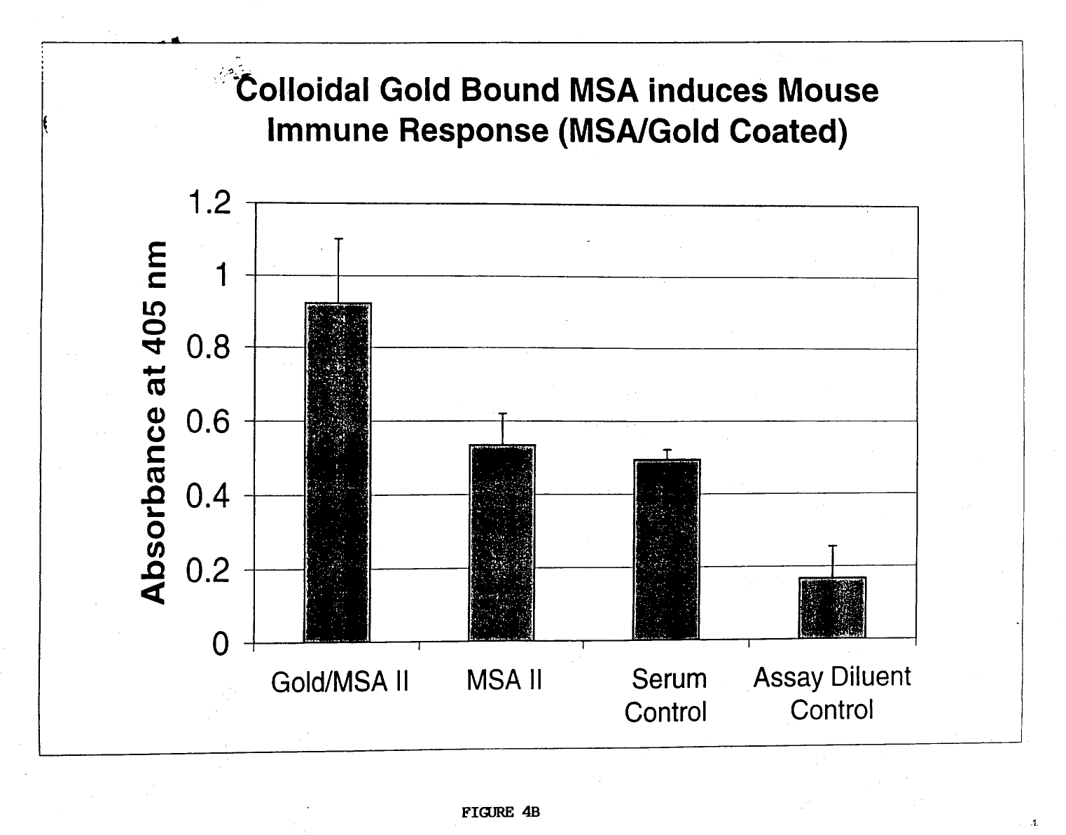

Briefly, polystyrene 96-well plate (Nunc) was coated with 100 μl of either free MSA or colloidal gold-bound MSA at 10 μg/ml in coating buffer at 4° C. overnight. Additional binding sites were blocked with 1% BSA in TBS for 2 hrs at 37° C. The plates were washed five times with washing buffer (0.2% Tween 20 in PBS), incubated with diluted antisera (1:100 dilution) or pooled control sera for 2 hrs at RT, washed five times with washing buffer, and incubated with goat anti-mouse IgG conjugated with alkaline phosphatase (1:1000 dilution) for 2 hrs at RT. After six washes with washing buffer, a substrate solution (2 mg/ml p-nitrophenyl phosphate in substrate buffer containing 10% diethanolamine, 100 μg/ml MgCL₂.6H₂O, pH 9.8) was added to each well. The color development was read with a microplate reader at 405 nm. The results from two separate experiments were summarized in FIGS. 4A and 4B. Immunization with the colloidal gold-mouse serum albumin (MSA) complex resulted in enhanced mouse immune response to MSA. Since MSA is a mouse protein that should be tolerated by the experimental animals, the data strongly support the potential of inducing autoimmunity using colloidal metal-protein complexes.

### EXAMPLE 6

### Preparation of P815 Cells for Conjugation with Colloidal Gold

P815 cells were cultured in DMEM supplemented with 10% FBS and antibiotics (penicillin and streptomycin) at 37° C. and 5% CO₂ to 80% confluency. The cells were harvested and counted. Cell viability was determined with trypan blue staining procedure.

### EXAMPLE 7

### Effect of pH on P815 Cell Loses and Colloidal Gold Flocculation

The objective of this experiment was to find out the optimal pH for colloidal gold particles binding to P815 cells. Different aliquots (10 ml each) of colloidal gold suspension were dialyzed (mwco 10,000-12,000 dialysis tube) against borax buffers with pH=6, pH=7, pH=8, pH=9, and pH=10, respectively. Each 5 ml of dialyzed colloidal gold suspension was then mixed with 5 million of P815 cells (>95% viability) for 2 hrs at 4° C. The degree of cell loses was determined under a microscope. The degree of colloidal gold flocculation was assessed by measurement of absorption spectra, or by visual scoring of color change. Among the buffers tested, the borax buffer with pH=8 gave the least degree of cell lysis and colloidal gold flocculation. In addition, there was a noticeable decrease of cell lysis when cells were first suspended in DMEM without phenol red (pH=8), then mixed with colloidal gold at a ratio of 1:3 (DMEM: colloidal gold).

### EXAMPLE 8

### Immunization of DBA/2 mice with P815-Colloidal Gold Conjugates

In general, since cancer cells, such as melanocytes and melanoma cells, may tolerize T-cell in vivo [Matzinger, Annu Rev Immunol, 12:991-1045, (1994)], repeated immunization may be necessary to induce complete regression of tumors.

Female DBA/2 mice (Charles River Laboratories, New York) at 6-8 weeks of age were maintained in a standard condition and were allowed to adapt their environment for one week before experiments. Mice were immunized three times at intervals of 10 days. There were four different immunization preparations: 1) P815 cell lysate, 2) colloidal gold-bound P815 alone, 3) colloidal gold-bound P815 with codon modified murine GMCSF (cGMCSF) DNA, and 4) colloidal gold-bound P815 with CFA/IFA, as shown in Table 1.

P815 cell lysate was used for the control group. Each mouse was injected (i.p.) with 100 μl of lysate which was equivalent to 10⁷ P815 cells. P815 cells lysate were prepared as follows: 10⁸ cells were lysized in 5 ml of deionized water overnight and frozen at −80° C. for 2 hrs, then lyophilized. The lysate pellet was reconstituted in saline for injection. cGMCSF DNA (100 μg in saline) was intramuscularly (i.m.) administered after P815-colloidal gold antigens were injected i.p. CFA/IFA (100 μl per injection per animal) was emulsified with P815-colloidal gold antigens before immunizations. Animals were administered with CFA in the first injection and with IFA in booster injection.

### EXAMPLE 9

### Preparation of P815 cells for Challenging Immunized DBA Mice.

P815 cells were cultured in DMEM supplemented with 10% FBS and antibiotics (penicillin and streptomycin), at 37° C. and 5% CO₂ to 90% confluence. Cells were harvested and their viability was measured with trypan blue staining procedure. Cells were washed three times with PBS and resuspended in saline.

### EXAMPLE 10

### Immunization with P815-Colloidal Gold conjugates inhibit P815 induced tumor growth in DBA mice.

As shown in Table 1,

TABLE 1

| Group | Mouse #1 | Mouse #2 | Mouse #3 |
|---|---|---|---|
| P815 Cell Lysate | 600 mm³ | 648 mm³ | 480 mm³ |
| P815-Colloidal Gold | 150 mm³ | 324 mm³ | Tumor |
| | | | disappear |
| P815-Colloidal Gold + cGMCSF | 120 mm³ | 60 mm³ | Tumor |
| | | | disappear |
| P815-Colloidal Gold + CFA/IFA | No tumor | No tumor | No tumor |
| | formed | formed | formed |

animals were challenged with 10 million P815 live cells (a minimal tumorigenic dose of P815 was reported to be 2×10³ cells) [Schmidt et al., PNAS 94:3262-3267, (1997)]. Tumor sizes were measured after 26 days of the initial P815 challenge. One mouse was free from tumor in the P815-Colloidal Gold+cGMCSF group, and all three mice were free from tumor in the P815-Colloidal Gold+CFA/IFA group. The tumor free-mouse in the P815-Colloidal Gold+cGMCSF group was rechallenged twice with 10⁶ and 10⁷ P815 cells, respectively. The tumor free mice in the P815-Colloidal Gold+CFA/IFA group was rechallenged five times with P815 cells, respectively. After initial challenge which is day 0, challenges were given at the follwing days: at day 23 (2×10⁷ of P815 cells at back-neck, s.c.); day 64 (10⁶ at the top of tail-base, s.c.); day 79 (10⁷ at the top of tail-base, s.c.); day 129 (10⁷ at the top of tail-base, s.c.); and day 196 (2×10⁵ at the top of tail-base, s.c.).

Table 1 demonstrated that CFA/IFA adjuvant and cGMCSF with colloidal gold-bound P815 membrane proteins induce a long-last specific protection of animals from subsequent tumor challenges.

As shown in Table 2, 3 and 5, mice were immunized three times at day 0 (first injection), day 9 (second injection) and day 21 (third injection). There were six different immunization preparations:

1) KLH alone, each mouse was injected with 100 μg of KLH in 100 μl of saline.

2) cGMCSF alone, cGMCSF DNA was mixed with gene transfection liposome (Vaxim) in a ratio of 1:5. Each animal was injected i.m. with 100 μg of cGMCSF.

3) P815-Gold/KLH, colloidal gold-bound P815 cells (equivalent to 10⁷ P815 cells for one injection per mouse), were emulsified with 100 μg of KLH and injected i.p. 4) P815-Gold/cGMCSF, colloidal gold-bound P815 cells (equivalent to 10⁷ P815 cells for one injection per one mouse), were injected i.p., whereas cGMCSF prepared as discussed in preparation 2 was injected i.m.

5) P815-Gold/KLH/cGMCSF, colloidal gold-bound P815 cells were emulsified and injected as discussed in preparation 3. The mice of cGMCSF group were injected as discussed in preparations 2 and 4.

6) P815-Gold/HA, colloidal gold-bound P815 cells were emulsified with 100 μg of hyaluronic acid (HA, Sigma) and injected i.p.

At day 27, each DBA mouse was subcutaneously challenged with 5×10⁶ P815 cells in 100 μl of saline on top of tail-base of the mouse. The dose of P815 cells used in each challenge was 2500 times higher than the reported tumorigenic dose of P815 (2×10³). Twenty-three days after challenge, tumor volume was measured as the product of the larger diameter by the smaller diameter by the thickness, and was expressed in mm³.

TABLE 2
Volume of Tumor (mm³, 23 days post P815 challenge)

| TREATMENTS | #1 MOUSE | #2 MOUSE | #3 MOUSE |
|---|---|---|---|
| KLH Alone | 330 | 168 | 12 |
| cGMCSF Alone | 504 | 294 | 288 |
| P815-Gold/KLH | 450 | 432 | no tumor |
| P815-Gold/cGMCSF | 880 | 352 | 20 |
| P815-Gold/KLH/cGMCSF | 384 | 210 | 15 |
| P815-Gold/HA | 240 | 80 | 24 |

TABLE 3
Volume of Tumor (mm³, 28 days post P815 challenge)

| TREATMENTS | #1 MOUSE | #2 MOUSE | #3 MOUSE |
|---|---|---|---|
| KLH Alone | 1020 | 308 | 120 |
| cGMCSF Alone | 1122 | 759 | 374 |
| P815-Gold/KLH | 462 | 220 | no tumor |
| P815-Gold/cGMCSF | 1040 | 572 | 40 |
| P815-Gold/KLH/cGMCSF | 450 | 360 | 60 |
| P815-Gold/HA | 198 | 56 | no tumor |

TABLE 4
Volume of Tumor (mm³, 37 days of P815 challenge)

| TREATMENTS | #1 MOUSE | #2 MOUSE | #3 MOUSE |
|---|---|---|---|
| KLH Alone | died | 1200 | 561 |
| cGMCSF Alone | died | died | 1248 |
| P815-Gold/KLH | died | died | no tumor |
| P815-Gold/cGMCSF | died | 720 | 220 |
| P815-Gold/KLH/cGMCSF | died | 672 | 216 |
| P815-Gold/HA | died | 210 | no tumor |

TABLE 5
Volume of Tumor (mm³, 60 days of P815 challenge)

| TREATMENTS | #1 MOUSE | #2 MOUSE | #3 MOUSE |
|---|---|---|---|
| KLH Alone | died | died | died |
| cGMCSF Alone | died | died | died |
| P815-Gold/KLH | died | died | 84 |
| P815-Gold/cGMCSF | died | died | died |
| P815-Gold/KLH/cGMCSF | died | died | 840 |
| P815-Gold/HA | died | 800 | no tumor |

### EXAMPLE 11

### Co-Culture of DBA Mouse Spleen Cells with P815 Cells.

Preparation of Spleen Cells: DBA mice were injected at day 1 with KLH alone, P815-gold/KLH, or P815-gold/HA as described in Example 10. The mice were challenged with P815 cells on day 27 and were sacrificed on day 70. DBA mouse spleens were removed, washed three times with RPMI 1640 (Biofluids), and compressed through sterile nylon mesh with a rubber stopper. Harvested cells from each spleen were centrifuged for 10 min in a sorval H-1000B rotor at 2,000 rpm and resuspended in 5 ml of RPMI 1640 supplemented with 10% FBS and antibiotics (penicillin and streptomycin). Cell viability was determined by trypan blue exclusion. Spleen cells (1 ml) from different treatment groups were plated in a 24-well tissue culture plate with a cell population of 5×10⁶ per well. The cells were cultured at 37° C., 5% CO₂ for 24 hrs.

Preparation of P815 Cells: P815 cells were cultured in DMEM supplemented with 10% FBS and antibiotics (penicillin and streptomycin) at 37° C. and 5% CO₂ until 90% confluence. Cells were harvested and cell viability was measured with trypan blue staining procedure. Cells were washed three times with PBS, and then, resuspended in RPMI 1640 complete medium. Each number of P815 cells (in 1 ml RPMI 1640 complete medium) were added to each well of 24 well plate that contained the precultured spleen cells. There were three replicate wells for each number of P815 cells. The spleen cells and P815 cells were co-cultured at 37° C. and 5% CO₂ for 24 hrs. Culture supernatants were collected and the concentrations of IFN-γ were determined by ELISA.

### EXAMPLE 12

### In Vitro IFN-γ Assay

Anti-mouse IFN-γ (R46A2; monoclonal antibody, Pharmingen) was diluted in coating buffer (pH=9.6) and coated (5 μg protein/well) onto 96-well ELISA plates (Nunc Immunoplates; Nunc. Corp., Naperville, Ill.) by incubation at 4° C. overnight. The plates were washed 6 times in washing buffer, then blocked with PBS containing 0.05% Tween 20 (Sigma), 1% low-fat dry milk (Carnation; Nestlé Food Corp., Glendale, Calif.), and 4% goat serum (KPL Laboratories, Gaithersburg, Md.) for 1 hr. After washing, the plates were incubated with samples diluted in PBS containing 1% BSA and 0.05% Tween 20 for 1 hr. The plates were incubated with rabbit anti-mouse IFN-γ (CytImmune Sciences, Inc. College, Md.) diluted 1:1000 in the same PBS solution for 1 hr and washed 6 times. The goat anti-rabbit IgG alkaline phosphatase conjugate was added to plates and incubated for 30 min. The bound mouse IFN-γ was quantitated by colorimetric reaction using p-nitrophenyl phosphate as a substrate (KPL Laboratories). The optical density (OD) was measured at 405 nm in a Bio-Tek Plate Reader (Winooski, Vt.). All samples and dilutions were tested in duplicate. For these assays, the limit of detection was 3 pg of IFN-γ per ml.

### EXAMPLE 13

### Enhanced IFN-γ Secretion of Spleen Cells from P815-Colloidal Gold Immunized DBA Mice.

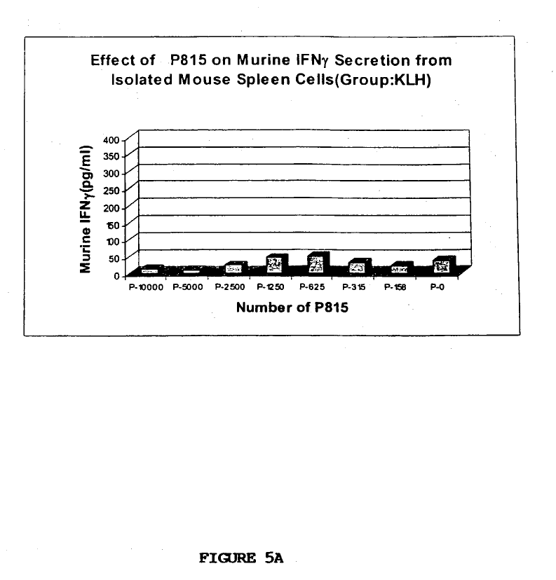

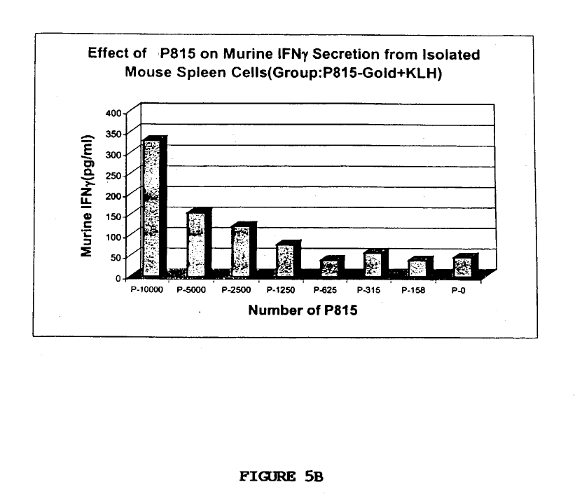

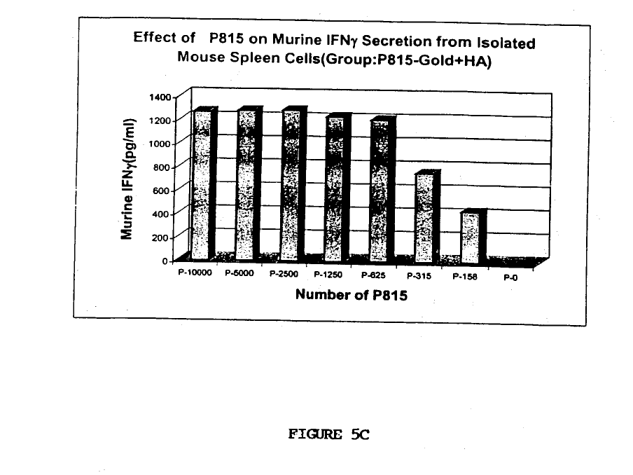

FIGS. 5A-5C illustrate the effect of P815 on murine IFN-γ secretion from isolated mouse spleen cells. Each figure represents a typical response of IFN—γ in vitro secretion from isolated spleen cells from three mice.

X-axis represents number of P815 cells co-cultured with 5×10⁶ spleen cells. For example, P-10000 equals 10,000 of P815 cells; P-0 means there is no P815 cells co-cultured with spleen cells. These results demonstrate that immunization with gold-P815 complexes significantly enhances the anti-P815 immune responses in DBA/2 mice.

It is to be understood that the above detailed examples are intended to illustrate and not limit the scope of the invention. Other aspects, advantages and modifications within the scope of the invention will be apparent to those skilled in the art to which the invention pertains.

What is claimed is:

1. A method for prevention and/or treatment of a tumor in a subject, said method comprising the steps of:
a) contacting colloidal metal with a tumor cell in an aqueous solution, wherein said tumor cell is isolated from said subject, and wherein said colloidal metal binds to a cell membrane protein of said tumor cell to form a colloidal metal-protein complex,
b) separating said colloidal metal-protein complex from other cellular components, and
c) administering a therapeutically effective amount of said colloidal metal-protein complex to said subject.

2. The method for prevention and/or treatment of a tumor according to claim 1, wherein said colloidal metal-protein complex is administered with a pharmaceutically acceptable carrier.

3. The method for prevention and/or treatment of a tumor according to claim 1, wherein said colloidal metal-protein complex is administered with an adjuvant.

4. The method for prevention and/or treatment of a tumor according to claim 3, wherein said adjuvant is selected from the group consisting of KLH, cGM-CSF, GM-CSF and HA.

5. The method for prevention and/or treatment of a tumor according to claim 1, wherein said colloidal metal-protein complex is capable of inducing an immune response to a self-antigen.

6. The method for prevention and/or treatment of a tumor according to claim 1, wherein said metal is select from the group consisting of gold, silver, platinum, titanium, vanadium, chromium, manganese, cobalt, gallium, strontium, tungsten, rhenium, gadolinium, aluminum, ruthenium, zinc, iron, nickel and calcium.

7. The method for prevention and/or treatment of a tumor according to claim 6, wherein said metal is gold.

8. A method of making a composition for stimulating an immune response in a mammal, said method comprising the steps of:
contacting colloidal metal with a cell in an aqueous solution, wherein said colloidal metal binds to a cell membrane protein of said cell to form a colloidal metal-protein complex, and
separating said colloidal metal-protein complex from other cellular components.

9. The method according to claim 8, wherein said aqueous solution has a pH of 9.0.

10. The method according to claim 8, wherein said cell is a tumor cell.

11. The method according to claim 8, wherein said metal is select from the group consisting of gold, silver, platinum, titanium, vanadium, chromium, manganese, cobalt, gallium, strontium, tungsten, rhenium, gadolinium, aluminum, ruthenium, zinc, iron, nickel and calcium.

12. The method according to claim 11, wherein said metal is gold.

13. The method according to claim 8, further comprising a step of obtaining said tumor cell from said mammal.

14. A composition for stimulating an immune response against a tumor in a mammal, said composition is made by:
contacting colloidal metal with a tumor cell in an aqueous solution, wherein said tumor cell is isolated from said mammal, and wherein said colloidal metal binds to a cell membrane protein of said tumor cell to form a colloidal metal-protein complex, and
separating said colloidal metal-protein complex from other cellular components.

15. A pharmaceutical composition for stimulating an immune response in a mammal comprising the composition of claim 14 and a pharmaceutically acceptable carrier.

16. The pharmaceutical composition according to claim 15, further comprising an adjuvant.

17. The pharmaceutical composition according to claim 16, wherein said adjuvant is selected from the group consisting of KLH, cGM-CSF, GM-CSF and HA.

18. The pharmaceutical composition according to claim 15, wherein said composition is capable of inducing an immune response to a self-antigen.

19. The composition according to claim 14, wherein said metal is gold.

20. A method for prevention and/or treatment of a tumor in a human, said method comprising administering to said human a therapeutically effective amount of said composition of claim 15.
